(19) United States
(12) Patent Application Publication
(10) Pub. No.: US 2026/0053762 A1
(43) Pub. Date: Feb. 26, 2026

(54) USE OF PDH INHIBITOR FOR TREATING NEUROPATHY

(71) Applicant: DONG-A UNIVERSITY RESEARCH FOUNDATION FOR INDUSTRY-ACADEMY COOPERATION, Busan (KR)

(72) Inventors: Jae Hyeon KIM, Busan (KR); Kyong Hwa KANG, Busan (KR); Ji Hyun LEE, Busan (KR); Hyong Jong KOH, Busan (KR)

(21) Appl. No.: 19/335,835

(22) Filed: Sep. 22, 2025

Related U.S. Application Data
(63) Continuation-in-part of application No. PCT/KR2024/003473, filed on Mar. 20, 2024.

(30) Foreign Application Priority Data
Mar. 23, 2023 (KR) 10-2023-0037813

(51) Int. Cl. A61K 31/192; A61P 25/02

(52) U.S. Cl. CPC A61K 31/192; A61P 25/02

(57) ABSTRACT

The present invention reveals that PDH can be utilized as a new target for the treatment of neuropathy and, based on this finding, provides preventive and therapeutic agents for neuropathy. Additionally, the present invention provides a method for screening candidate substances for neuropathy prevention and treatment. The screening method of the present invention allows for the effective selection of new candidate substances that can prevent and/or treat neuropathy, and the substances selected by the screening method can be used to provide a pharmaceutical composition for the prevention and treatment of neuropathy.

### TECHNICAL FIELD

The present application claims priority to Korean Patent Application No. 10-2023-0037813, filed on Mar. 23, 2023, and the entire disclosure of the aforementioned application is incorporated herein by reference.

The present invention relates to the use of an inhibitor of PDH (pyruvate dehydrogenase) for treating neuropathy, and more specifically, to a composition for preventing, improving, or treating neuropathy comprising an inhibitor of expression or activity of PDH as an active ingredient, and a method for screening the same.

### BACKGROUND ART

Neuropathy develops either due to direct damage to nerve tissues or diseases that affect nerve tissues, and it can be classified into sensory neuropathy, motor neuropathy, and autonomic neuropathy depending on the type of affected nerve tissue. Various factors have been suggested as causes of peripheral neuropathy, which is a type of the aforementioned neuropathy, including genetic disorders, metabolic and endocrine system disorders, inflammatory diseases, nutritional deficiencies such as vitamin deficiency, and anticancer agents administered during cancer treatment.

Among these, chemotherapy-induced peripheral neuropathy (CIPN) is distinct from cancer pain caused by direct invasion of nerve tissues by cancer, nerve compression, or bone metastasis. It is observed in approximately 40% of patients undergoing combination chemotherapy. CIPN may persist for several months to years even after the completion of cancer treatment, and in severe cases, it can last a lifetime, negatively affecting the quality of life of patients. It is also distinguished from physiological pain (acute pain) such as general pain or inflammatory pain.

As such, chemotherapy-induced peripheral neuropathy negatively impacts the quality of life of cancer patients, but currently, there are no effective preventive measures or established treatments. Depending on the severity of symptoms, adjustments to the chemotherapy schedule or dose reduction are considered, and in severe cases, switching to another anticancer agent is taken into account.

In the past, studies have been conducted using amifostine, anticonvulsants, antidepressants, vitamins, CaMg minerals, acetyl-L-carnitine, and omega-3 fatty acids to prevent the occurrence of CIPN, but significant effects compared to control groups have not been demonstrated. In terms of treatment for CIPN, tricyclic antidepressants such as amitriptyline and nortriptyline, or anticonvulsants such as gabapentin, have not significantly improved patients'symptoms. However, since these drugs have shown effectiveness in other types of neuropathies unrelated to anticancer agents and have mild side effects, they are currently used in clinical practice for chemotherapy-related peripheral neuropathy.

However, these drugs have inherent side effects such as dizziness and drowsiness, and special caution is required for elderly patients as tricyclic antidepressants may cause side effects. Additionally, due to their low safety margin, high-dose administration is not feasible, which is a limitation.

### Problem to be Solved

Accordingly, the inventors of the present invention have conducted comprehensive studies to develop a drug usable for the treatment of neuropathy and discovered that a substance inhibiting the expression or activity of PDH can achieve such effects, thereby completing the present invention.

Therefore, an object of the present invention is to provide a pharmaceutical composition for preventing or treating neuropathy comprising an inhibitor of expression or activity of PDH (pyruvate dehydrogenase) as an active ingredient.

Another object of the present invention is to provide a pharmaceutical composition for preventing or treating neuropathy consisting of an inhibitor of expression or activity of PDH (pyruvate dehydrogenase).

Another object of the present invention is to provide a pharmaceutical composition for preventing or treating neuropathy consisting essentially of an inhibitor of expression or activity of PDH (pyruvate dehydrogenase).

Another object of the present invention is to provide a food composition for preventing or improving neuropathy comprising an inhibitor of expression or activity of PDH (pyruvate dehydrogenase) as an active ingredient.

Another object of the present invention is to provide a food composition for preventing or improving neuropathy consisting of an inhibitor of expression or activity of PDH (pyruvate dehydrogenase).

Another object of the present invention is to provide a food composition for preventing or improving neuropathy consisting essentially of an inhibitor of expression or activity of PDH (pyruvate dehydrogenase).

Another object of the present invention is to provide a method for screening candidate substances for preventing and treating neuropathy comprising: (a) treating cells expressing mRNA or protein of PDH with a test substance; (b) measuring mRNA expression level of PDH, protein expression level of PDH, or activity of PDH protein; and (c) selecting a substance that reduces mRNA expression level of PDH, protein expression level of PDH, or activity of PDH protein compared to a control group (untreated group) as a candidate substance.

Another object of the present invention is to provide the use of an inhibitor of expression or activity of PDH for manufacturing a composition for treating neuropathy.

Another object of the present invention is to provide a method for treating neuropathy comprising administering an effective amount of a composition comprising an inhibitor of expression or activity of PDH as an active ingredient to a subject in need thereof.

### Means for Solving the Problem

To achieve the aforementioned objectives of the present invention, the present invention provides a pharmaceutical composition for preventing or treating neuropathy comprising an inhibitor of expression or activity of PDH (pyruvate dehydrogenase) as an active ingredient.

To achieve the objectives of the present invention, the present invention provides a pharmaceutical composition for preventing or treating neuropathy consisting of an inhibitor of expression or activity of PDH (pyruvate dehydrogenase).

To achieve another objective of the present invention, the present invention provides a pharmaceutical composition for preventing or treating neuropathy essentially consisting of an inhibitor of expression or activity of PDH (pyruvate dehydrogenase).

To achieve another objective of the present invention, the present invention provides a food composition for preventing or improving neuropathy comprising an inhibitor of expression or activity of PDH (pyruvate dehydrogenase) as an active ingredient.

To achieve another objective of the present invention, the present invention provides a food composition for preventing or improving neuropathy consisting of an inhibitor of expression or activity of PDH (pyruvate dehydrogenase).

To achieve another objective of the present invention, the present invention provides a food composition for preventing or improving neuropathy essentially consisting of an inhibitor of expression or activity of PDH (pyruvate dehydrogenase).

To achieve another objective of the present invention, the present invention provides a method for screening candidate substances for preventing and treating neuropathy comprising the following steps: (a) treating cells expressing mRNA or protein of PDH with a test substance; (b) measuring mRNA expression level of PDH, protein expression level of PDH, or activity of PDH protein; and (c) selecting a substance that reduces mRNA expression level of PDH, protein expression level of PDH, or activity of PDH protein compared to a control group (untreated group) as a candidate substance.

To achieve another objective of the present invention, the present invention provides the use of an inhibitor of expression or activity of PDH for manufacturing a composition for treating neuropathy.

To achieve another objective of the present invention, the present invention provides a method for treating neuropathy comprising administering an effective amount of a composition comprising an inhibitor of expression or activity of PDH as an active ingredient to a subject in need thereof.

Hereinafter, the present invention will be described in detail.

The present invention provides a pharmaceutical composition for preventing or treating neuropathy comprising an inhibitor of expression or activity of PDH (pyruvate dehydrogenase) as an active ingredient.

In one embodiment, the neuropathy may be selected from the group consisting of diabetic neuropathy, postherpetic neuropathy, trigeminal neuropathy, peripheral neuropathies, mononeuropathy, mononeuritis multiplex, autonomic neuropathy, inflammatory neuropathy, metabolic or endocrine neuropathy, toxic or drug-induced neuropathy, nutritional neuropathy, vasculitic neuropathy, paraneoplastic neuropathy, traumatic neuropathy, hereditary neuropathy, and idiopathic neuropathy, and preferably, the neuropathy may be drug-induced neuropathy.

In one embodiment, the drug-induced neuropathy may be chemotherapy-induced peripheral neuropathy (CIPN).

In one embodiment, the anticancer agent may be at least one selected from the group consisting of platinum series, taxane series, vinca alkaloids, bortezomib, and thalidomide.

In one embodiment, the inhibitor of PDH expression may be siRNA (small interference RNA), shRNA (short hairpin RNA), miRNA (microRNA), ribozyme, DNAzyme, PNA (peptide nucleic acids), antisense oligonucleotide, or aptamer that complementarily binds to DNA or mRNA of PDH gene.

In one embodiment, the inhibitor of PDH activity may be a compound, peptide, peptide mimetics, fusion protein, antibody, or aptamer that specifically binds to PDH protein.

In one embodiment, the inhibitor of PDH activity may be CPI-613 (Devimistat), PDK (PDH kinase), an enhancer of expression or activity of PDK, or an inhibitor of expression or activity of PDP (PDH phosphatase).

In one embodiment, the enhancer of PDK expression may be DJ-1 or an enhancer of its expression, or HIF-1 (hypoxia-inducible factor 1) or an enhancer of its expression.

In the present specification, the term “inhibitor of expression or activity of PDH or PDP” refers to a substance that directly or indirectly acts on PDH or PDP to reduce or inhibit the expression or activity of PDH or PDP. Such substances may include compounds, proteins, fusion proteins, antibodies, amino acids, peptides, viruses, carbohydrates, lipids, nucleic acids, extracts, fractions, and the like, without limitation. The mechanism by which the substance inhibits the expression or activity of PDH or PDP is not particularly limited and may act, for example, by reducing gene expression such as transcription or translation or by converting the active form into an inactive form.

In the present specification, the term “enhancer of expression of PDK, DJ-1, or HIF-1” refers to a substance that directly or indirectly acts on PDK, DJ-1, or HIF-1 to improve, induce, stimulate, or increase the expression of PDK, DJ-1, OR HIF-1, without limitation.

The mechanism by which the substance promotes the expression of PDK, DJ-1, or HIF-1 is not particularly limited and may act, for example, by increasing gene expression such as transcription or translation or by converting the inactive form into an active form.

In the present specification, the term “recombinant vector” refers to a vector designed to express PDK, DJ-1, or HIF-1, and preferably includes liposomes, plasmid vectors, cosmid vectors, bacteriophage vectors, virus vectors, and the like, but not limited thereto, as long as the vector can express PDK, DJ-1, or HIF-1 in vivo. Examples of the virus vectors include adenovirus, adeno-associated virus, retrovirus, lentivirus, herpes simplex virus, alphavirus, and the like.

In the present specification, the term “prevention” refers to any act of suppressing or delaying the onset of neuropathy through the administration of the composition according to the present invention.

In the present specification, the term “treatment” refers to any act of improving or beneficially altering the symptoms of neuropathy and its complications through the administration of the composition according to the present invention. A person skilled in the art can determine the exact criteria for diseases for which the composition of the present invention is effective and assess the degree of improvement, enhancement, and treatment by referring to materials presented by organizations such as the Domestic Medical Association.

In the present specification, the term “improvement” refers to any act of at least reducing parameters related to the treated condition, such as the severity of symptoms. At this time, the composition of the present invention may be used simultaneously or separately with therapeutic agents for the prevention or improvement of neurodegenerative diseases.

The therapeutically effective amount of the composition of the present invention may vary depending on various factors such as the method of administration, target site, and condition of the subject.

The pharmaceutical composition of the present invention is administered in a pharmaceutically effective amount. The term “pharmaceutically effective amount” as used in the present invention refers to an amount sufficient to treat the disease with a reasonable benefit/risk ratio applicable to medical treatment and without causing side effects. The effective dosage level may be determined based on factors such as the health condition of the subject, the type and severity of the neurodegenerative disease, the activity of the drug, the sensitivity to the drug, the method of administration, the time of administration, the route of administration, the excretion rate, the treatment period, and the combination or simultaneous use of other drugs, as well as other factors well known in the medical field. The composition of the present invention may be administered as a single therapeutic agent or in combination with other therapeutic agents, and may be administered sequentially or simultaneously with conventional therapeutic agents, either as a single or multiple doses. Considering all the above factors, it is important to administer the minimum amount necessary to achieve maximum effect without side effects, which can be easily determined by those skilled in the art.

In one embodiment, the pharmaceutical composition may be one or more formulations selected from the group consisting of oral formulations, topical agents, suppositories, sterile injectable solutions, and sprays.

The composition of the present invention may also include carriers, diluents, excipients, or combinations of two or more thereof commonly used in biological preparations. The pharmaceutically acceptable carrier is not particularly limited as long as it is suitable for in vivo delivery of the composition, and may include, for example, compounds known in the art, saline, sterile water, Ringer's solution, buffered saline, dextrose solution, maltodextrin solution, glycerol, ethanol, and mixtures of one or more of these components, and may further include other conventional additives such as antioxidants, buffers, and bacteriostatic agents as needed. Additionally, diluents, dispersants, surfactants, binders, and lubricants may be added to formulate aqueous solutions, suspensions, emulsions, pills, capsules, granules, or tablets. Furthermore, the composition may be formulated according to each disease or component using appropriate methods in the art or methods known in the art.

The composition of the present invention may further contain one or more active ingredients exhibiting the same or similar functions. The composition of the present invention comprises the active ingredient in an amount of 0.0001 to 10% by weight, preferably 0.001 to 1% by weight, based on the total weight of the composition.

The pharmaceutical composition of the present invention may further include pharmaceutically acceptable additives, and the pharmaceutically acceptable additives may include starch, pregelatinized starch, microcrystalline cellulose, lactose, povidone, colloidal silicon dioxide, dicalcium phosphate, lactose, mannitol, malt, gum arabic, pregelatinized starch, corn starch, powdered cellulose, hydroxypropyl cellulose, Opadry, sodium starch glycolate, carnauba wax, synthetic aluminum silicate, stearic acid, magnesium stearate, aluminum stearate, calcium stearate, sucrose, dextrose, sorbitol, and talc, among others. The pharmaceutically acceptable additives according to the present invention are preferably included in an amount of 0.1 to 90 parts by weight relative to the composition, but are not limited thereto.

The composition of the present invention may be administered parenterally (e.g., intravenously, subcutaneously, intraperitoneally, or topically) or orally, and oral administration is most preferred. The dosage may vary depending on the body weight, age, gender, health condition, diet, time of administration, method of administration, excretion rate, and severity of the disease of the subject.

Liquid formulations for oral administration of the composition of the present invention include suspensions, solutions, emulsions, and syrups, which may include various excipients such as wetting agents, sweeteners, flavoring agents, and preservatives in addition to simple diluents such as water and liquid paraffin. Formulations for parenteral administration include sterile aqueous solutions, non-aqueous solvents, suspensions, emulsions, freeze-dried formulations, and suppositories.

In the present invention, the content of the composition is not particularly limited depending on the intended use or aspect, and may be, for example, 0.01 to 99% by weight, preferably 0.5 to 50% by weight, and more preferably 1 to 30% by weight, based on the total weight of the composition. Additionally, the pharmaceutical composition according to the present invention may further include additives such as pharmaceutically acceptable carriers, excipients, or diluents in addition to the active ingredient. The pharmaceutical composition of the present invention may include the compound prepared by the method of the present invention in an amount of 0.1 to 99.9% by weight and a carrier in an amount of 99.9% to 0.1% by weight.

The present invention also provides a food composition for preventing or improving neuropathy comprising an inhibitor of expression or activity of PDH (pyruvate dehydrogenase) as an active ingredient.

The food composition of the present invention includes all forms such as functional food, nutritional supplements, health food, and food additives.

The food composition of the above type may be prepared in various forms according to conventional methods known in the art. Without limitation, for example, health foods may be prepared in the form of tea, juice, and drinks containing the active ingredient, and may be consumed in liquid, granulated, encapsulated, or powdered form. Additionally, the active ingredient may be mixed with known active ingredients effective for improving skin conditions and prepared in the form of a composition. Furthermore, functional foods may include, but are not limited to, beverages (including alcoholic beverages), fruits and their processed foods (e.g., canned fruits, bottled fruits, jams, marmalades, etc.), fish, meat, and their processed foods (e.g., ham, sausage, corned beef, etc.), bread and noodles (e.g., udon, soba, ramen, spaghetti, macaroni, etc.), fruit juices, various drinks, cookies, taffy, dairy products (e.g., butter, cheese, etc.), edible vegetable oils, margarine, vegetable proteins, retort foods, frozen foods, various seasonings (e.g., soybean paste, soy sauce, sauces, etc.), to which the active ingredient is added. Additionally, the active ingredient may be prepared in powder or concentrate form for use as an additive.

The preferred content of the active ingredient in the food composition of the present invention is not limited thereto but is preferably 0.1 to 90% by weight in the final prepared food. More preferably, the food composition of the present invention may be prepared in the form of functional food or dietary supplements, particularly mixed with active ingredients known to have effects on preventing or improving neuropathy.

The present invention also provides a method for screening candidate substances for preventing and treating neuropathy comprising the following steps: (a) treating cells expressing mRNA or protein of PDH with a test substance; (b) measuring mRNA expression level of PDH, protein expression level of PDH, or activity of PDH protein; and (c) selecting a substance that reduces mRNA expression level of PDH, protein expression level of PDH, or activity of PDH protein compared to a control group (untreated group) as a candidate substance.

The substance that reduces the activity of PDH may be siRNA, shRNA, miRNA, ribozyme, DNAzyme, PNA (peptide nucleic acids), antisense oligonucleotide, peptide, antibody specific to PDH protein, aptamer, natural extract, or chemical substance, but is not limited thereto and includes all substances used or to be used in the future to inhibit the expression of genes and/or proteins in the art.

In the present invention, the measurement of mRNA expression level of PDH may be performed using RT-PCR, quantitative or semi-quantitative RT-PCR, quantitative or semi-quantitative real-time RT-PCR, northern blot, and DNA or RNA chip, but is not limited thereto.

In the present invention, the measurement of protein expression level of PDH may be performed using any one method selected from the group consisting of western blot, ELISA, radioimmunoassay, radial immunodiffusion, Ouchterlony immunodiffusion, rocket immunoelectrophoresis, immunohistochemical staining, immunoprecipitation assay, complement fixation assay, FACS, and protein chip, but is not limited thereto.

After step (c) in the present invention, a step of evaluating pharmacological activity in an appropriate in vivo model may be further performed.

The present invention also provides the use of an inhibitor of expression or activity of PDH for manufacturing a composition for treating neuropathy.

The present invention also provides a method for treating neuropathy comprising administering an effective amount of a composition comprising an inhibitor of expression or activity of PDH as an active ingredient to a subject in need thereof.

The “effective amount” in the present invention refers to an amount that, when administered to a subject, exhibits an effect of improving, treating, detecting, diagnosing, or suppressing or reducing neuropathy or neuropathy-related conditions. The “subject” may be an animal, preferably a mammal, particularly an animal including humans, and may also include cells, tissues, organs, etc., derived from animals. The subject may be a patient in need of the effect.

The “treatment” in the present invention comprehensively refers to improving symptoms caused by neuropathy or the disease, and may include curing, substantially preventing, or improving the condition, and includes alleviating, curing, or preventing one or most symptoms caused by the disease, but is not limited thereto.

In the present specification, the term “comprising” is used in the same sense as “including” or “characterized by” and does not exclude additional components or steps not specifically mentioned in the composition or method according to the present invention. The term “consisting of” excludes additional elements, steps, or components not separately described. The term “essentially consisting of” means that the scope of the composition or method may include substances or steps that do not substantially affect its basic characteristics in addition to the described substances or steps.

### Effect of the Invention

The present invention reveals that PDH can be utilized as a new target for the treatment of neuropathy and, based on this, provides a preventive and therapeutic agent for neuropathy. Additionally, it provides a method for screening candidate substances for preventing and treating neuropathy. According to the screening method of the present invention, new candidate substances capable of preventing and/or treating neuropathy can be effectively selected, and by using the substances selected according to the screening method of the present invention, it is possible to provide a pharmaceutical composition for preventing and treating neuropathy.

### BRIEF DESCRIPTION OF THE DRAWINGS

FIG. 1a is a result showing the overexpression of the PDK gene (ppk>PDK), which inhibits PDH, in sensory neurons of a Drosophila model sensitized to heat probe stimulation by the anticancer agent paclitaxel.

FIG. 1b is a result confirming that the sensitivity of a Drosophila model sensitized to heat probe stimulation by the anticancer agent paclitaxel is normalized when a mutation is induced in the PDP gene (PDP^{P}), which activates PDH.

FIG. 2 is a result confirming that the sensitivity of a Drosophila model sensitized to heat probe stimulation by the administration of the anticancer agent paclitaxel is normalized when CPI-613 is co-administered.

FIG. 3a is a schematic diagram illustrating the experimental process for observing changes in sensitivity to von Frey filament stimulation caused by the administration of the anticancer agent paclitaxel.

FIG. 3b is a result confirming that the sensitivity of a mouse model sensitized to von Frey filament stimulation by the administration of the anticancer agent paclitaxel is normalized when CPI-613 is co-administered.

### DETAILED DESCRIPTION OF THE INVENTION

Hereinafter, the present invention will be described in detail with reference to the following examples. However, the following examples are merely illustrative of the present invention, and the present invention is not limited thereto.

### Experimental Methods

### 1. PDK Gene Overexpression and PDP Gene Mutation Drosophila Model

To specifically express the Drosophila PDK gene in sensory neurons, a Drosophila model (ppk>PDK) expressing the PDK gene in sensory neurons was created by crossing ppk-GAL4 Drosophila (ppk), which specifically expresses the yeast transcription factor in Drosophila sensory neurons, with UAS-PDK Drosophila, which has a transgene where the Drosophila PDK gene is linked to UAS, a specific promoter of GAL4. For the PDP gene mutation Drosophila, PDP^{P} Drosophila, in which the expression of the PDP gene is inhibited by the insertion of p-element, a transposon of Drosophila, into the PDP gene, was used. Wild-type Drosophila w¹¹¹⁸ (Con) was used as a control group.

### 2. Paclitaxel-Induced Peripheral Neuropathy Drosophila Model

After mating Drosophila for 48-72 hours, eggs were collected and cultured in standard Drosophila medium (1.5 g yeast+2.1 g glucose+0.15 g agar+1.125 g cornmeal per 30 ml) for 72 hours to obtain third instar (L3) larvae. Drug media were prepared by adding four combinations of drugs to the standard Drosophila medium: Control (DMSO), Paclitaxel 10 μM, Paclitaxel 10 μM+CPI-613 5 mM, and CPI-613 5 mM. After washing the L3 larvae with distilled water, they were transferred to each drug medium and cultured for 48 hours, followed by measurement of larval thermal nociception.

### 3. Larval Thermal Nociception Assays

After washing L3 larvae (120 hours post-oviposition) with distilled water, they were acclimated in a petri dish for 10 seconds. The abdominal A4-A5 segments of the larvae were stimulated with a thermal probe, 0.6 mm in width, controlled at 40° C. by a microprocessor. The time required to induce a specific curling response to thermal nociception for each larva was measured and recorded. Larvae that did not exhibit a curling response within 20 seconds were considered non-responsive. At least 30 larvae per group were analyzed, and the average time required to induce a curling response and the statistical significance of the differences in average times between groups were calculated using GraphPad Prism 9 software.

### 4. Paclitaxel-Induced Peripheral Neuropathy Mouse Model

Nine-week-old female C57BL6 mice were obtained, and mice with sensory abnormalities were excluded through von-Frey testing before drug administration. PBS (control), paclitaxel (16 mg/kg) dissolved in PBS, and CPI-613 (10 mg/kg, 20 mg/kg) were administered intraperitoneally according to the drug combinations and schedules shown in the figure. CPI-613 was administered 1 hour prior to paclitaxel administration. On the 15th day after completing drug administration, the mechanical allodynia response of each mouse was measured using von-Frey testing.

### 5. Measurement of Mechanical Allodynia in Mice (Von-Frey Test)

A transparent plastic box (10×10 cm) with closed sides except for the bottom was placed on a wire mesh (3×3 mm), and the experimental animals were allowed to acclimate for about 20 minutes. A series of eight stiff nylon von-Frey monofilaments (3.61, 3.84, 4.08, 4.31, 4.56, 4.74, 4.93, and 5.18 mN, Stoelting, Wood Dale, IL, USA; equivalent in grams to 0.4, 0.6, 1.0, 2.0, 4.0, 6.0, 8.0, and 15.0) was used, and the up-down method with a 50% threshold value was applied to evaluate the pain response. The experiment was conducted by first identifying the most sensitive area of both hind paws using the middle filament of 2.0 g (4.31 mN) among the eight von-Frey filaments, applying enough force to bend the filament for 3-4 seconds. If no paw withdrawal response was observed, the experiment was repeated with a filament of one step higher intensity. If a response was observed, the sensitivity of the initially selected area was evaluated by stimulating it with a weaker filament. Each stimulus was applied at 2-second intervals to minimize the influence of the previous stimulus.

### Experimental Results

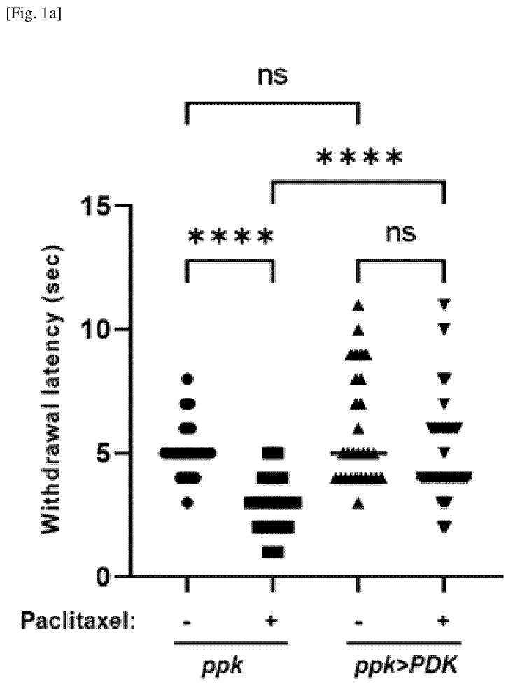

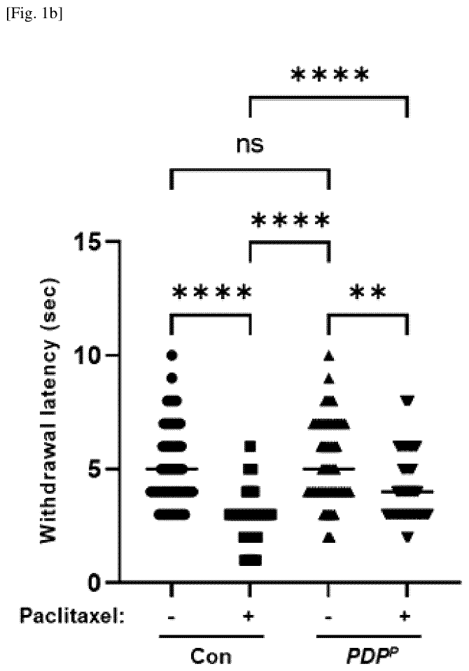

As shown in FIGS. 1a and 1b, it was confirmed that the sensitivity of the Drosophila model, which became sensitive to heat probe stimulation due to the anticancer drug paclitaxel, was normalized when the PDK gene, which inhibits PDH, was overexpressed in sensory neurons (ppk>PDK) (FIG. 1a) or when a mutation was induced in the PDP gene, which activates PDH (PDP^{P}) (FIG. 1b).

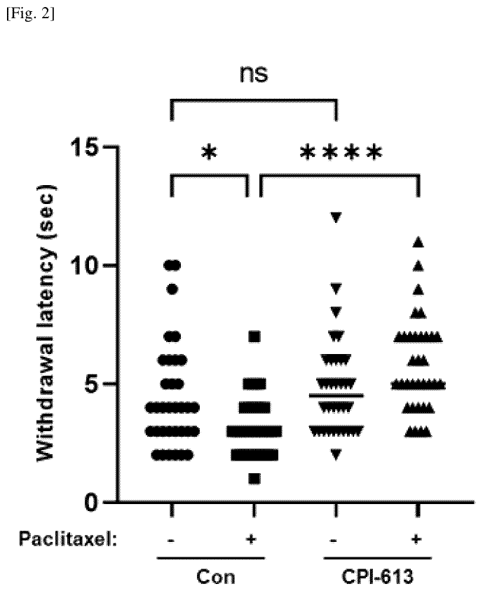

As shown in FIG. 2, it was confirmed that the sensitivity of the Drosophila model, which became sensitive to heat probe stimulation due to paclitaxel administration, was normalized when the PDH inhibitor CPI-613 was co-administered.

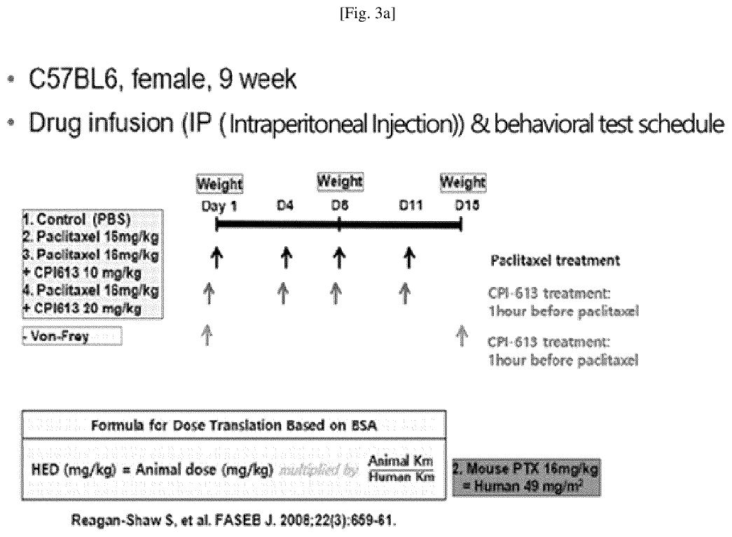

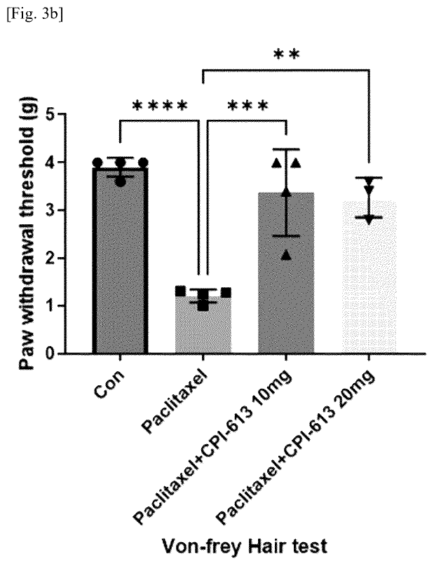

As shown in FIG. 3b, it was confirmed that the sensitivity of the mouse model, which became sensitive to von-Frey filament stimulation due to paclitaxel administration, was normalized when CPI-613 was co-administered.

### INDUSTRIAL APPLICABILITY

The present invention reveals that PDH can be utilized as a new target for the treatment of neuropathy and, based on this, provides a preventive and therapeutic agent for neuropathy. Additionally, it provides a method for screening candidate substances for preventing and treating neuropathy. According to the screening method of the present invention, new candidate substances capable of preventing and/or treating neuropathy can be effectively selected, and by using the substances selected according to the screening method of the present invention, it is possible to provide a pharmaceutical composition for preventing and treating neuropathy, thereby having high industrial applicability.

What is claimed is:

1. A pharmaceutical composition for preventing or treating neuropathy comprising an inhibitor of expression or activity of PDH (pyruvate dehydrogenase) as an active ingredient.

2. The pharmaceutical composition of claim 1, wherein the neuropathy is selected from the group consisting of diabetic neuropathy, postherpetic neuropathy, trigeminal neuropathy, peripheral neuropathies, mononeuropathy, mononeuritis multiplex, autonomic neuropathy, inflammatory neuropathy, metabolic or endocrine neuropathy, toxic or drug-induced neuropathy, nutritional neuropathy, vasculitic neuropathy, paraneoplastic neuropathy, traumatic neuropathy, hereditary neuropathy, and idiopathic neuropathy.

3. The pharmaceutical composition of claim 2, wherein the drug-induced neuropathy is chemotherapy-induced peripheral neuropathy (CIPN).

4. The pharmaceutical composition of claim 3, wherein the anticancer agent is at least one selected from the group consisting of platinum series, taxane series, vinca alkaloids, bortezomib, and thalidomide.

5. The pharmaceutical composition of claim 1, wherein the inhibitor of PDH expression is siRNA (small interference RNA), shRNA (short hairpin RNA), miRNA (microRNA), ribozyme, DNAzyme, PNA (peptide nucleic acids), antisense oligonucleotide, or aptamer that complementarily binds to DNA or mRNA of PDH gene.

6. The pharmaceutical composition of claim 1, wherein the inhibitor of PDH activity is a compound, peptide, peptide mimetics, fusion protein, antibody, or aptamer that specifically binds to PDH protein.

7. The pharmaceutical composition of claim 1, wherein the inhibitor of PDH activity is CPI-613 (Devimistat), PDK (PDH kinase), an enhancer of expression or activity of PDK, or an inhibitor of expression or activity of PDP (PDH phosphatase).

8. The pharmaceutical composition of claim 7, wherein the enhancer of PDK expression is DJ-1 or an enhancer of its expression, or HIF-1 (hypoxia-inducible factor 1) or an enhancer of its expression.

9. A method for screening candidate substances for preventing and treating neuropathy comprising:
(a) treating cells expressing mRNA or protein of PDH with a test substance;
(b) measuring mRNA expression level of PDH, protein expression level of PDH, or activity of PDH protein; and
(c) selecting a substance that reduces mRNA expression level of PDH, protein expression level of PDH, or activity of PDH protein compared to a control group (untreated group) as a candidate substance.

10. The screening method of claim 9, wherein the measurement of mRNA expression level of PDH is performed using any one method selected from the group consisting of RT-PCR, quantitative or semi-quantitative RT-PCR, quantitative or semi-quantitative real-time RT-PCR, northern blot, DNA chip and RNA chip.

11. The screening method of claim 9, wherein the measurement of protein expression level of PDH is performed using any one method selected from the group consisting of western blot, ELISA, radioimmunoassay, radial immunodiffusion, Ouchterlony immunodiffusion, rocket immunoelectrophoresis, immunohistochemical staining, immunoprecipitation assay, complement fixation assay, FACS, and protein chip.

12. A food composition for preventing or improving neuropathy comprising an inhibitor of expression or activity of PDH (pyruvate dehydrogenase) as an active ingredient.

13. Use of an inhibitor of expression or activity of PDH for manufacturing a composition for treating neuropathy.

14. A method for treating neuropathy comprising administering an effective amount of a composition comprising an inhibitor of expression or activity of PDH as an active ingredient to a subject in need thereof.
